(19) United States
(12) Patent Application Publication
(10) Pub. No.: US 2023/0142776 A1
(43) Pub. Date: May 11, 2023

(54) PARENT-CHILD HEALTH MANAGEMENT SYSTEM, PARENT-CHILD HEALTH ANALYSIS DEVICE, AND PARENT-CHILD HEALTH MANAGEMENT METHOD

(71) Applicants: NATIONAL UNIVERSITY CORPORATION HOKKAIDO UNIVERSITY, Sapporo-shi, Hokkaido (JP); MORINAGA MILK INDUSTRY CO., LTD., Tokyo (JP); Hitachi, Ltd., Tokyo (JP)

(72) Inventors: Takahiro NAKAMURA, Tokyo (JP); Takashi TAKEMOTO, Tokyo (JP); Masanori YOSHINO, Tokyo (JP); Tokiyoshi AYABE, Sapporo-shi, Hokkaido (JP); Takashi KIMURA, Sapporo-shi, Hokkaido (JP); Hirohisa IZUMI, Kanagawa (JP); Fuka TABATA, Kanagawa (JP)

(21) Appl. No.: 17/910,935

(22) PCT Filed: Mar. 12, 2021
(86) PCT No.: PCT/JP2021/010012

§ 371 Date: Sep. 12, 2022

(30) Foreign Application Priority Data
Jun. 19, 2020 (JP) 2020-106029

(51) Int. Cl. G16H 10/20

(52) U.S. Cl. CPC G16H 10/20

(57) ABSTRACT

An analysis device in a parent-child health management system receives a sensor output from a sensor terminal and an answer to a questionnaire from a user terminal. At least one of parent's life information and child's life information is analyzed based on the answer to the questionnaire to generate a life information analysis result. A sensor output analysis unit generates, based on the sensor output, a sensing result including at least one of a parent-child relationship, a parent's health condition, and a child's health condition. A health estimation unit is configured to compare the life information analysis result and the sensing result with a predetermined criterion to estimate at least one of a parent-child state, a parent's state, and a child's state. An output unit is configured to select a questionnaire and advice corresponding to the estimated state and output the selected questionnaire and advice to the user terminal.

### CROSS-REFERENCE TO RELATED APPLICATION(S)

The present application claims priority from Japanese Patent Application No. 2020-106029 filed on Jun. 19, 2020, contents of which are incorporated into the present application by reference.

### TECHNICAL FIELD

The present invention relates to a system for analyzing and managing health of a parent and a child.

### BACKGROUND ART

For a purpose of assisting healthy development of children, a mother-child health examination is performed to examine lifestyles and health conditions of mothers during pregnancy and after childbirth and children from newborns to school children.

The following related art is described as background art in the present technical field. PTL 1 (JP-A-2011-108142) discloses a profiling device. In the profiling device, an output screen generation unit generates an output screen for displaying question information, and displays the output screen on a user terminal to present the output screen to a user. An information acquisition unit acquires answer information indicating an answer of the user to the question information and biological information of the user. An information storage unit stores the question information, the answer information, and the biological information corresponding to the answer information in association with each other. An answer determination unit determines whether to re-question based on the biological information of the user corresponding to the answer information. When the answer determination unit determines to re-question, the output screen generation unit generates an output screen for displaying re-question information, and displays the output screen on the user terminal to present the output screen to the user (with reference to the abstract).

### CITATION LIST

### Patent Literature

PTL 1: JP-A-2011-108142

### SUMMARY OF INVENTION

### Technical Problem

The above-described mother-child health examination is an examination for a long period of time, and thus some mothers and children withdraw from the examination. Although questionnaires are effective in acquiring lifestyle information of mothers and children, questionnaires on dietary habits and the like are more troublesome as compared to advantages, due to complexity of answers, making it difficult to continuously acquire information. In particular, since predetermined questions are delivered at predetermined times and the number of questions becomes large, participants feel troublesome to answer them, so keeping participants from leaving and reducing the withdrawal rate is required.

Therefore, an object of the invention is to provide a solution for continuously acquiring information from a parent and a child as an examination target and reducing a withdrawal rate of the examination.

### Solution to Problem

A representative example of the invention disclosed in the present application is as follows. That is, a parent-child health management system comprising: a sensor terminal configured to sense at least one of a parent's state and a child's state; a user terminal to which data related to at least one of parent's life and child's life is input; and an analysis device configured to receive data collected by the sensor terminal and the user terminal. The analysis device is implemented by a computer including an arithmetic device, which executes predetermined arithmetic processing to implement the following functional units, and a storage device accessible by the arithmetic device. The analysis device includes: a reception unit configured to receive a sensor output from the sensor terminal and an answer to a questionnaire input to the user terminal; a questionnaire analysis unit configured to analyze at least one of parent's life information and child's life information based on the answer to the questionnaire and generate a life information analysis result; a sensor output analysis unit configured to generate, based on the sensor output, a sensing result including at least one of a parent-child relationship, a parent's health condition, and a child's health condition; a health estimation unit configured to compare the life information analysis result and the sensing result with a predetermined criterion to estimate at least one of a parent-child state, a parent's state, and a child's state; and an output unit configured to select a questionnaire and advice corresponding to the estimated state and output the selected questionnaire and advice to the user terminal.

### Advantageous Effects of Invention

According to an aspect of the invention, it is possible to reduce a burden on a user who answers a questionnaire and reduce a withdrawal rate of an examination. Problems, configurations, and effects other than those described above are clarified with the following description of embodiments.

### BRIEF DESCRIPTION OF DRAWINGS

FIG. 1 is a diagram showing an example of a configuration of a parent-child health analysis device according to a first embodiment.

FIG. 2 is a diagram showing an example of a configuration of a questionnaire table according to the first embodiment.

FIG. 3 is a diagram showing an example of a configuration of an advice table according to the first embodiment.

FIG. 4 is a diagram showing an example of a configuration of a parent-child health analysis device according to a second embodiment.

FIG. 5 is a diagram showing reading of a barcode of a product according to the second embodiment.

### DESCRIPTION OF EMBODIMENTS

Hereinafter, embodiments of a parent-child health management system of the invention is described with reference to the drawings.

### First Embodiment

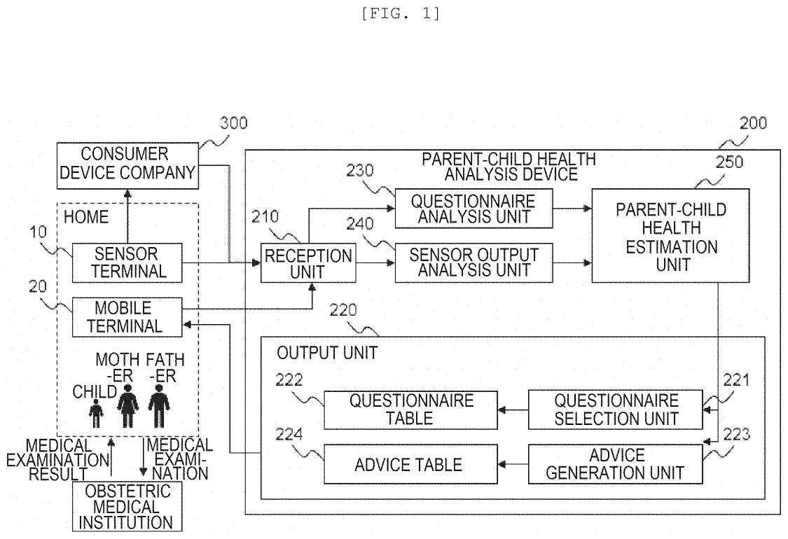

FIG. 1 is a diagram showing an example of a configuration of a parent-child health analysis system according to a first embodiment.

The parent-child health management system according to the first embodiment includes a parent-child health analysis device 200, a sensor terminal 10, and a mobile terminal 20. The parent-child health analysis device 200 is connected to the sensor terminal 10 that senses a home living space, and the mobile terminal 20 that is used by a family having a parent (at least one of a father and a mother) and a child as members. In the following description, members of the family are referred to as users when the members are not distinguished from each other.

The sensor terminal 10 is a sensor installed in a living space such as a home, detects a state of a user, and transmits a sensor output. The sensor terminal 10 is, for example, a camera that captures an image of a living space, a microphone (for example, an AI speaker or a smart speaker) that detects a sound of the living space, a wearable device that measures an activity amount or a heartbeat of the user wearing the sensor terminal 10, or a sphygmomanometer worn by the user. The activity amount of the user can be calculated based on an acceleration measured by the wearable device. In addition, an activity amount of a user who is sleeping or an infant on a bed may be measured using a mat-shaped activity meter laid on a bed. As the sensor terminal 10, a name tag type device capable of simultaneously acquiring an acceleration, a voice, and a face-to-face relationship between sensor terminals may be used.

Since the sensor terminal 10 acquires data of the user in the living space, from a viewpoint of privacy protection, the sensor terminal 10 may anonymize the acquired data, convert a camera image into a skeleton model, or acquire data of only the user who has agreed to use.

The mobile terminal 20 is a terminal, such as a smartphone or a tablet terminal, used by a user. The user inputs an answer to a questionnaire using the mobile terminal 20. For example, a father or a mother inputs an answer (for example, selecting one or more from choices or texting answer to the questionnaire) to a questionnaire related to parenting. Instead of the mobile terminal 20, a personal computer, a bidirectional television, or the like may be used.

The parent-child health analysis device 200 is connected to the sensor terminal 10 and the mobile terminal 20 via a network such as a wide area network (WAN) and a local area network (LAN). A connection method of the network may be either wired or wireless. The parent-child health analysis device 200 may be directly connected to the sensor terminal 10, or may be connected to the sensor terminal 10 via a server 300 of a consumer device company (for example, an IT platform that sells smart speakers).

The parent and child have a medical examination at a medical institution such as obstetrics and gynecology to acquire a medical examination result.

The parent-child health analysis device 200 includes a reception unit 210, an output unit 220, a questionnaire analysis unit 230, a sensor output analysis unit 240, and a parent-child health estimation unit 250, and analyzes parent-child health by executing a predetermined program.

The reception unit 210 receives, from the sensor terminal 10, a sensor output that detects a state of the user at home, and outputs the sensor output to the sensor output analysis unit 240. In addition, the reception unit 210 receives an answer to a questionnaire input by the user from the mobile terminal 20, and outputs the answer to the questionnaire analysis unit 230. That is, the answer to the questionnaire is input to the mobile terminal 20 in a form of an answer number to an option and a text answer to a question, and is transferred to the questionnaire analysis unit 230 via the reception unit 210.

The questionnaire analysis unit 230 analyzes the received answer to the questionnaire and outputs a life information analysis result. The life information analysis result may include information, such as a meal content or a meal time of the parent and/or the child, which cannot be acquired by the sensor terminal 10. In addition, the answer is analyzed by using a statistical method in terms of the number and a ratio of non-input items and a tendency of the answer, and other aspects. In addition, a time point at which the answer to the questionnaire is received is analyzed. The time point may be classified into one hour unit or a time period such as morning, noon, or night.

In addition, the questionnaire analysis unit 230 may compare the answer input by the user with a predetermined answer request criterion to determine validity of the answer. Specifically, it is preferable to determine whether the number of answers to the questionnaire or an answer rate reaches a statistically valid value and a reliable result is acquired. In addition, it may be estimated that all users whose answers to the questionnaire are medium (3 of options at 5 stages from 1 to 5) do not accurately answer the questionnaire, and the result of the questionnaire may be excluded. In addition, the answer input by the user may be compared with the answer tendency. Specifically, abnormality of the questionnaire answer may be detected based on whether there is a discrepancy with the previous answer related to a similar question, whether there is a discrepancy in the answer with another user who has answered the same questionnaire, or the like.

The sensor output analysis unit 240 analyzes the sensor output, which is input from the sensor terminal 10 to the reception unit 210, and outputs a sensing result. The sensing result includes at least one of a parent-child relationship, a parent's health condition, and a child's health condition. For example, a parent and a child are recognized from a camera image as the sensor output, and a distance between the parent and the child is calculated. In addition, a length of time the child cries is measured by a voice acquired by a microphone. In addition, a parent's state and a child's state (during action, during sleep, or the like) are estimated based on activity amounts of the parent and the child measured by the wearable device. In addition, based on heart rates of the parent and the child measured by the wearable device, a time (a time point, a time period, or the like) when the parent and the child are relaxed and a time (a time point, a time period, or the like) when the parent and the child are tensed are calculated.

The parent-child health estimation unit 250 comprehensively analyzes the answer to the questionnaire and the sensor output, and estimates a parent-child health condition and the parent-child relationship. The health condition and the relationship each have a plurality of indices, and a numerical value of each index is calculated. The numerical value of each index may be a binary value, or may be determined by any decimal number from 0 to 1. The parent-child health estimation unit 250 determines whether the health condition and the relationship correspond to a label by comparing the calculated numerical value of each index with a predetermined threshold. The label is a state corresponding to at least one of the parent's health condition, the child's health condition, and the parent-child relationship, and is, for example, postpartum depression, allergy, asthma, underweight, or developmental retardation. Specifically, regarding a label of postpartum depression, in a case where it is determined that a distance between the parent and the child is longer than a predetermined distance and a duration time when the parent and the child are separated is longer than a predetermined time, a degree of closeness between the parent and the child is low (for example, a time when the parent and the child are in close contact with each other is shorter than a predetermined threshold), and the mother does not take good care of the child, it can be estimated that there is a possibility of neglect or postpartum depression. In addition, presence or absence of allergy in the child may be estimated based on a questionnaire answer related to food. In addition, underweight of the child may be estimated based on a questionnaire answer related to a medical examination result (height, weight). Furthermore, a degree of development may be estimated based on an activity amount of the child and a situation of utterance.

The output unit 220 includes a questionnaire selection unit 221, a questionnaire table 222, an advice generation unit 223, and an advice table 224.

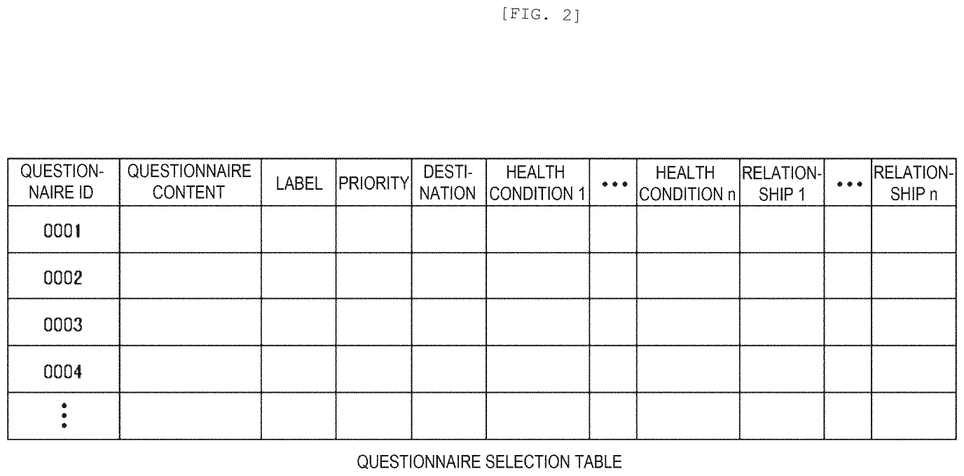

The questionnaire selection unit 221 determines a questionnaire to be transmitted to the user based on the health condition and the relationship estimated by the parent-child health estimation unit 250 with reference to the questionnaire table 222 shown in FIG. 2. The questionnaire transmitted to the user includes questions related to medical examination results necessary for estimating the parent-child health condition, a state of daily life, meal contents of the parent and the child, or the like. In the questionnaire table 222, conditions including the estimated health condition and the relationship and the questionnaire transmitted to the user are recorded in association with each other. Among items of the questionnaire table 222, the label indicates the parent-child state determined based on the estimated health condition and relationship. As described above, the label includes postpartum depression, allergy, asthma, underweight, developmental retardation, or the like. The parent-child health estimation unit 250 determines whether the parent-child state corresponds to the label as an estimation result. The questionnaire selection unit 221 may determine whether the parent-child state corresponds to the label based on the estimation result of the parent-child health estimation unit 250.

In the questionnaire table 222, a questionnaire content is determined according to the label, and includes, for example, “when did you start eating food A?”, “do you enjoy living with your child?”, or the like. A priority is an index used to narrow down the questionnaire to be presented or determine an order of presenting the questionnaire when a plurality of questionnaires are transmitted to the user. For example, when a situation needs to be dealt with early or when it is a beginning of an event that is likely to become serious, a high priority is set to the questionnaire to be transmitted to the user. The priority may be set for each label. A destination is the destination to transmit in order to acquire a valid answer in the questionnaire. For example, the questionnaire is normally transmitted to a mother, but when postpartum depression is estimated, there is a possibility that a valid answer cannot be acquired even if the questionnaire is transmitted to the mother. Therefore, the questionnaire may be transmitted to the father to acquire a valid answer. In addition, without using the questionnaire table 222, the questionnaire may be determined by multiple regression analysis using the estimated health condition and relationship as explanatory variables, or the questionnaire may be selected using a neural network by artificial intelligence.

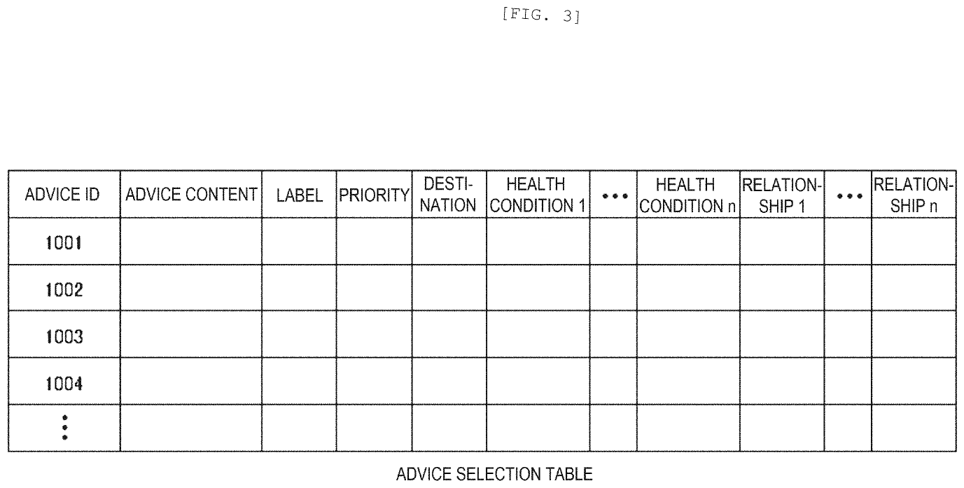

The advice generation unit 223 determines advice to be transmitted to the user based on the health condition and the relationship estimated by the parent-child health estimation unit 250 with reference to the advice table 224 shown in FIG. 3. The advice generation unit 223 may determine advice related to the questionnaire selected by the questionnaire selection unit 221. The advice transmitted to the user includes life advice for improving the health of the parent and the child, advice related to food recommended to be ingested based on the parent-child health condition, or the like. In the advice table 224, conditions including the estimated health condition and relationship and the advice transmitted to the user are recorded in association with each other. In addition, without using the advice table 224, the advice may be determined by multiple regression analysis using the estimated health condition and relationship as explanatory variables, or the advice may be selected using a neural network by artificial intelligence. Contents of the advice table 224 may be added or corrected by a system administrator.

The parent-child health estimation unit 250 may not be provided. In this case, the questionnaire selection unit 221 determines a questionnaire to be transmitted to the user based on the analysis results of the questionnaire analysis unit 230 and the sensor output analysis unit 240. For example, the questionnaire may be determined by multiple regression analysis using the estimated health condition and relationship as explanatory variables, or the questionnaire may be selected using a neural network by artificial intelligence. In addition, the advice generation unit 223 determines advice to be transmitted to the user based on the analysis results of the questionnaire analysis unit 230 and the sensor output analysis unit 240. For example, the advice may be determined by multiple regression analysis using the estimated health condition and relationship as explanatory variables, or the advice may be selected using a neural network by artificial intelligence.

The parent-child health analysis device 200 is implemented by a computer including a processor (CPU), a memory, an auxiliary storage device, and a communication interface, which are not shown. The parent-child health analysis device 200 may include an input and output interface for an operator to input and output data.

The processor is an arithmetic device that executes a program stored in the memory. By executing various programs by the processor, functions of the units (for example, the reception unit 210, the output unit 220, the questionnaire analysis unit 230, the sensor output analysis unit 240, the parent-child health estimation unit 250, or the like) of the parent-child health analysis device 200 are implemented. A part of the processing executed by the processor executing the program may be executed by another arithmetic device (for example, hardware such as an ASIC or an FPGA).

The memory includes a ROM, which is a non-volatile storage element, and a RAM, which is a volatile storage element. The ROM stores an invariable program (for example, a BIOS) or the like. The RAM is a high-speed and volatile storage element such as a dynamic random access memory (DRAM), and temporarily stores a program to be executed by the processor and data to be used when the program is executed.

The auxiliary storage device is a large-capacity, non-volatile storage device such as a magnetic storage device (for example, an HDD) and a flash memory (for example, an SSD). In addition, the auxiliary storage device stores data (for example, the questionnaire table 222, the advice table 224, or the like) used when the processor executes the program, and the program executed by the processor. That is, the program is read from the auxiliary storage device, loaded into the memory, and executed by the processor, thereby realizing each function of the parent-child health analysis device 200.

The communication interface is a network interface device that controls communication with other devices according to a predetermined protocol.

The input and output interface is an interface to which an input device such as a keyboard and/or a mouse is connected and which receives an input from an operator, but the input and output interface may correspond to a voice input or the like. The output interface is an interface to which an output device such as a display device or a printer (not shown) is connected and which outputs an execution result of the program in a format that can be visually recognized by the operator. A terminal connected to the parent-child health analysis device 200 via a network may provide the input device and the output device. In this case, the parent-child health analysis device 200 may have a function of a web server, and the terminal may access the parent-child health analysis device 200 using a predetermined protocol (for example, http).

The program executed by the processor of the parent-child health analysis device 200 is provided in the parent-child health analysis device 200 via a removable medium (a CD-ROM, a flash memory, or the like) or a network, and is stored in a non-volatile auxiliary storage device which is a non-transitory storage medium. Therefore, the parent-child health analysis device 200 may have an interface for reading data from the removable medium.

The parent-child health analysis device 200 may be a computer system implemented by one physical computer or by a plurality of logical or physical computers, or may be operated on a virtual computer constructed on a plurality of physical computer resources.

As described above, according to the first embodiment of the invention, the appropriate questionnaire and advice are selected and delivered based on the result of sensing the parent and the child and the health information and relationship of the parent and the child estimated from the questionnaire answer or the like. Therefore, only the questionnaire and the advice suitable for the parent and the child can be delivered to the mobile terminal 20, a burden on the user who answers the questionnaire can be reduced, and a withdrawal rate in the mother-child health examination can be reduced.

In addition, since the parent-child health estimation unit 250 comprehensively analyzes the answer to the questionnaire and the sensor output and determines the label (postpartum depression, allergy, asthma, underweight, developmental retardation, or the like) corresponding to the parent-child health condition and the parent-child relationship, it is possible to detect a sign of a state to be careful without being diagnosed in the medical institution. In addition, by using the label, it is possible to reliably detect a specific state to be careful.

### Second Embodiment

Next, a second embodiment of the invention will be described. In the parent-child health analysis device 200 according to the second embodiment, a parent-child master assigning unit 225 and a delivery timing control unit 226 are added to the above-described first embodiment. In the second embodiment, functions and configurations the same as those of the first embodiment are denoted by the same reference numerals, and description thereof will be omitted.

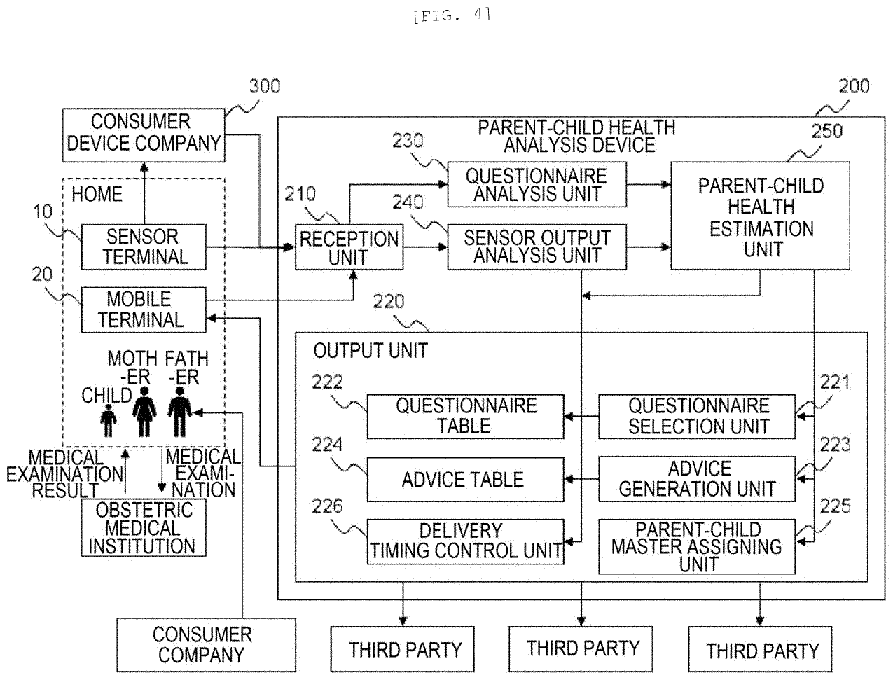

FIG. 4 is a diagram showing an example of a configuration of the parent-child health analysis device according to the second embodiment.

A parent-child health management system according to the second embodiment includes the parent-child health analysis device 200, the sensor terminal 10, and the mobile terminal 20. The parent-child health analysis device 200 is connected to the sensor terminal 10 and the mobile terminal 20 via a network. The parent-child health analysis device 200 includes the reception unit 210, the output unit 220, the questionnaire analysis unit 230, the sensor output analysis unit 240, and the parent-child health estimation unit 250. The output unit 220 includes the questionnaire selection unit 221, the questionnaire table 222, the advice generation unit 223, the advice table 224, the parent-child master assigning unit 225, and the delivery timing control unit 226.

The delivery timing control unit 226 controls a timing of delivering a questionnaire selected by the questionnaire selection unit 221 to the mobile terminal 20. In the method in the related art, since the questionnaire is delivered at a timing determined by a management side, when the delivery timing overlaps with a busy timing in childcare or the like, browsing or answering of the questionnaire becomes troublesome, an answer to the questionnaire becomes postponed, a possibility of withdrawing from the mother-child health examination becomes high, and continuation of the examination becomes difficult. Therefore, the delivery timing control unit 226 estimates a state of a user based on a sensor output from the sensor terminal 10, and controls the delivery timing so as to deliver the questionnaire in a time period in which the user have relatively enough time.

For example, the delivery timing control unit 226 may statistically process a daily activity amount of the user on a time axis, select a time period in which the activity amount is small while the user is awake, and deliver the questionnaire. In addition, a distance between a parent and a child may be statistically processed on the time axis, and the questionnaire may be delivered by selecting a time period in which a degree of closeness between the parent and the child is low. In addition, the questionnaire may be delivered at a time (for example, two hours before and after) close to a time when the questionnaire was answered in the past. In addition, the questionnaire may be delivered during a time when the sensed real-time activity amount is small while the user is awake. In addition, the questionnaire may be delivered in a time when a distance between the parent and the child is long in real time and the degree of closeness between the parent and the child is low. In addition, the questionnaire may be delivered at a timing when the child is not crying. Furthermore, a combination of these plurality of references may be used to deliver questionnaires, for example, in a time period of low activity amount in the past time and low parent-child relationship in real time.

Accordingly, by the delivery timing control unit 226 controlling the delivery timing of the questionnaire, it is possible to deliver the questionnaire at a time with reference to the user, to reduce a burden on the user, and to prevent withdrawal from the mother-child health examination.

The parent-child master assigning unit 225 gives a prize (award) to a family who meets a criterion for responding to the questionnaire. The criterion for giving an award is, for example, a case where inappropriate answers (the same answers to all items or contradictory answers) are few, the user answers seriously, the user continues to answer for a long time (for example, 5 years or longer), or the like. When an award is given, for example, top performers or ranking may be displayed in a community in which mother-child health examination subjects participate, a coupon for a product or a service may be given, or a service dedicated to the person may be provided. The output unit 220 may provide a third party company with information of the given award in order for the user to receive a superiority service or a dedicated service.

Accordingly, the parent-child master assigning unit 225 gives an award to the user, such that it is possible to give game performance and a profit of the user by ranking up to the answer to the questionnaire, and to increase motivation to the questionnaire answer by social contribution.

In addition, the output unit 220 according to the second embodiment may provide, in addition to the information of the award, information (so-called big data) acquired by a questionnaire or sensing to a third party company.

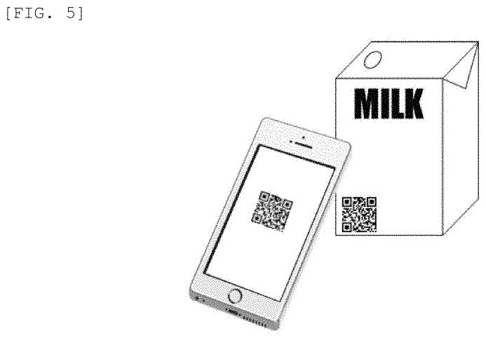

In addition, in the second embodiment, a questionnaire delivered by a consumer company (for example, food manufacturing and selling companies such as a dairy product manufacturing company and a confectionery manufacturing company) supporting the mother-child health examination may be processed by the parent-child health analysis device 200 according to the present embodiment. For example, as shown in FIG. 5, a link (for example, a two-dimensional barcode) for answering a questionnaire is displayed on a package of a product sold by a consumer food company. The user who purchased the product reads the link using the mobile terminal 20 and answers the questionnaire on an accessed answer page. Then, the questionnaire analysis unit 230 of the parent-child health analysis device 200 processes the answer. As described above, the consumer food company can guide the user to a unique questionnaire via a product, and can collect user evaluation for the product of the consumer food company.

The invention is not limited to the embodiments described above, and includes various modifications and equivalent configurations within the scope of the appended claims. For example, the embodiments described above has been described in detail for easy understanding of the invention, and the invention is not necessarily limited to those having all the configurations described above. In addition, a part of a configuration of a certain embodiment may be replaced with a configuration of another embodiment. In addition, the configuration of another embodiment may be added to the configuration of the certain embodiment. In addition, a part of the configuration of each embodiment may be added to, deleted from, and replaced with another configuration.

In addition, parts or all of the configurations, functions, processing units, processing methods described above or the like may be implemented by hardware, for example by designing with an integrated circuit, or may be implemented by software, with a processor to interpret and execute a program that implements each function.

Information such as a program, a table, a file, or the like that implements each function can be stored in a storage device such as a memory, a hard disk, and a solid state drive (SSD), or a recording medium such as an IC card, an SD card, and a DVD.

In addition, control lines and information lines indicate what is considered necessary for description, and not all the control lines and the information lines are necessarily shown in a product. In practice, it may be considered that almost all the configurations are connected with each other.

1. A parent-child health management system comprising:
a sensor terminal configured to sense at least one of a parent's state and a child's state;
a user terminal to which data related to at least one of parent's life and child's life is input; and
an analysis device configured to receive data collected by the sensor terminal and the user terminal, wherein
the analysis device is implemented by a computer including an arithmetic device, which executes predetermined arithmetic processing to implement the following functional units, and a storage device accessible by the arithmetic device, and
the analysis device includes
a reception unit configured to receive a sensor output from the sensor terminal and an answer to a questionnaire input to the user terminal,
a questionnaire analysis unit configured to analyze at least one of parent's life information and child's life information based on the answer to the questionnaire and generate a life information analysis result,
a sensor output analysis unit configured to generate, based on the sensor output, a sensing result including at least one of a parent-child relationship, a parent's health condition, and a child's health condition,
a health estimation unit configured to compare the life information analysis result and the sensing result with a predetermined criterion to estimate at least one of a parent-child state, a parent's state, and a child's state, and
an output unit configured to select a questionnaire and advice corresponding to the estimated state and to output the selected questionnaire and advice to the user terminal.

2. The parent-child health management system according to claim 1, wherein
the health estimation unit is configured to estimate, based on the sensing result, that there is a possibility that a parent is depressed when a degree of closeness between the parent and the child is lower than a predetermined threshold.

3. The parent-child health management system according to claim 1, wherein
the output unit includes a delivery timing control unit configured to deliver the selected questionnaire to the user terminal in a specified time period in which a parent has relatively enough time, based on the sensing result.

4. The parent-child health management system according to claim 1, wherein
the output unit includes a master assigning unit configured to give a prize to a user who meets a criterion for responding to the questionnaire.

5. The parent-child health management system according to claim 1, wherein
the output unit is configured to deliver a questionnaire to a user who is more likely to give a valid answer when there is a possibility of not receiving a valid answer to the questionnaire.

6. The parent-child health management system according to claim 1, wherein
the user terminal is configured to access a link by reading the link displayed on a product purchased by a user, and
the questionnaire analysis unit is configured to analyze a result of a questionnaire input by the user according to the link.

7. The parent-child health management system according to claim 1, wherein
the output unit includes
specifying labels corresponding to the parent-child state, the parent's state, and the child's state estimated by the health estimation unit,
selecting a questionnaire and advice to be transmitted to the user terminal according to the specified labels,
selecting one label according to priorities assigned to the labels, and
outputting a questionnaire and advice corresponding to the selected label.

8. A parent-child health analysis device implemented by a computer including an arithmetic device, which executes predetermined arithmetic processing to implement the following functional units, and a storage device accessible by the arithmetic device, wherein
a sensor terminal is configured to sense at least one of a parent's state and a child's state,
a user terminal is configured to receive an input of data related to at least one of parent's life and child's life, and
the parent-child health analysis device comprising:
a reception unit configured to receive a sensor output from the sensor terminal and an answer to a questionnaire input to the user terminal;
a questionnaire analysis unit configured to analyze at least one of parent's life information and child's life information based on the answer to the questionnaire and generate a life information analysis result;
a sensor output analysis unit configured to generate, based on the sensor output, a sensing result including at least one of a parent-child relationship, a parent's health condition, and a child's health condition;
a health estimation unit configured to compare the life information analysis result and the sensing result with a predetermined criterion to estimate at least one of a parent-child state, a parent's state, and a child's state; and
an output unit configured to select a questionnaire and advice corresponding to the estimated state and output the selected questionnaire and advice to the user terminal.

9. A parent-child health management method executed by a parent-child health management system, wherein
the parent-child health management system includes
a sensor terminal configured to sense at least one of a parent's state and a child's state,
a user terminal to which data related to at least one of parent's life and child's life is input, and
an analysis device configured to receive data collected by the sensor terminal and the user terminal and implemented by a computer including an arithmetic device, which executes predetermined arithmetic processing, and a storage device accessible by the arithmetic device, and
the parent-child health management method comprising:
a reception step of receiving, by the analysis device, a sensor output from the sensor terminal and an answer to a questionnaire input to the user terminal;
a questionnaire analysis step of analyzing at least one of parent's life information and child's life information based on the answer to the questionnaire and generating a life information analysis result by the analysis device;
a sensor output analysis step of generating, based on the sensor output, a sensing result including at least one of a parent-child relationship, a parent's health condition, and a child's health condition by the analysis device;
a health estimation step of comparing the life information analysis result and the sensing result with a predetermined criterion to estimate at least one of a parent-child state, a parent's state, and a child's state by the analysis device; and
an output step of selecting a questionnaire and advice corresponding to the estimated state and outputting the selected questionnaire and advice to the user terminal by the analysis device.
